(19) United States
(12) Patent Application Publication
(10) Pub. No.: US 2026/0335373 A1
(43) Pub. Date: Oct. 29, 2026

(54) TREATMENT OF CACHEXIA USING FIBROBLAST CELLS AND PRODUCTS THEREOF

(71) Applicant: FIBROBIOLOGICS, INC., Houston, TX (US)

(72) Inventors: Pete O'HEERON, Houston, TX (US); Thomas ICHIM, Houston, TX (US)

(21) Appl. No.: 19/564,134

(22) Filed: Mar. 11, 2026

Related U.S. Application Data
(63) Continuation of application No. 17/309,177, filed on May 3, 2021, now Pat. No. 12,589,119.

(51) Int. Cl. A61K 35/33; A61P 1/14; A61P 21/00; C12N 5/077

(52) U.S. Cl. CPC A61K 35/33; A61P 1/14; A61P 21/00; C12N 5/0656

(57) ABSTRACT

Embodiments of the disclosure concern methods and compositions related to treatment of cachexia or inflammation related thereto. In specific embodiments, cachexia or inflammation related thereto is to treated or prevented by the administration of fibroblasts. In specific embodiments, the fibroblasts are amniotic-derived fibroblasts.

This application is a continuation of U.S. patent application Ser. No. 17/309,177, filed May 3, 2021, which is a national phase application under 35 U.S.C. § 371 of International Application No. PCT/US2019/059678, filed Nov. 4, 2019, which claims priority to U.S. Provisional Patent Application Ser. No. 62/755,542, filed Nov. 4, 2018, all of which are incorporated by reference herein in their entirety.

### TECHNICAL FIELD

The present disclosure concerns at least the fields of cell biology, molecular biology, cell therapy, and medicine.

### BACKGROUND

Cachexia is a condition associated with muscle wasting particularly presenting in patients with cancer or chronic infections such as HIV, for example. Although studies have shown that inflammatory mediators are causative of cachexia, such as TNF-alpha, interventions to inhibit inflammation have been relatively ineffective in clinical situations. One potential reason for this disparity, is the multifactorial nature of cancer-associated inflammation.

The process of neoplastic transformation is associated with uncontrolled proliferation of cells, which gives rise to necrosis of de novo tissues, resulting in macrophage infiltration, complement activation, and neo-angiogenesis. In situations where this manifestation of neoplasia is occurring at a level where normal physiological processes are disturbed, systemic increases in inflammatory markers are observed, which give rise to exaggerated chronic inflammation that presents systemic consequences to the overall constitution of the patient.

The majority of cancer patients succumb to disease because of muscle wasting, metabolic abnormalities, and multiple organ failure. Although currently available interventions are focused on reducing tumor burden in a cancer patient, there is a need in the art for interventions which reduce pathologies associated with the impact of tumor masses on the general physiology of the patient. The present disclosure provides means for using fibroblasts to restore physiological, immunological and metabolic homeostasis in cancer patients, which allows for the possibility of innate and adaptive host responses to initiate, maintain, and execute activities which result in tumor stabilization and in some cases regression.

### BRIEF SUMMARY

Embodiments of the disclosure include methods for ameliorating cachexia, or inflammation related thereto, in an individual, comprising the steps of administering an effective amount of immune-modulating fibroblasts to the individual. The methods may also include culturing a population of fibroblasts under suitable conditions to induce production of immune-modulating activity, thereby producing the immune-modulating fibroblasts. The cachexia, or inflammation related thereto, may be initiated by a chronic inflammatory condition such as one selected from the group consisting of chronic infection, tuberculosis, HIV/AIDS, cancer, coeliac disease, chronic obstructive pulmonary disease, multiple sclerosis, rheumatoid arthritis, congestive heart failure, familial amyloid polyneuropathy, mercury poisoning (acrodynia), Crohn's disease, untreated/severe type 1 diabetes mellitus, anorexia nervosa, hormonal deficiency, and a combination thereof. In some cases, the chronic inflammatory condition is associated with the elevation of one or more detectable markers, such as one or more markers detected in the bloodstream. The individual may be known to have the chronic inflammatory condition or may be suspected of having or at risk for having a chronic inflammatory condition. Examples of detectable markers include those selected from the group consisting of C-reactive protein, Interleukin-1, Interleukin-6, TNF-alpha, and a combination thereof.

In particular embodiments of the disclosure, the fibroblasts utilized in methods are derived from amniotic fluid. Examples of fibroblasts derived from amniotic fluid may be those selected from the species from the group consisting of humans, primates, dogs, cats, goats, elephants, cattle, horses, pigs, mice, rabbits, and a combination thereof. In specific embodiments, the fibroblasts express detectable levels of one or more markers selected from the group consisting of SSEA3, SSEA4, Tra-1-60, Tra-1-81, Tra-2-54, Oct-4, CD13, CD44, CD49b, CD105, aminopeptidase N, and a combination thereof. In specific examples, the fibroblasts do not express detectable levels of SSEA1.

When an individual is provided fibroblasts, they may be administered to the individual affected by cachexia, or inflammation related thereto, by one or more routes selected from the group consisting of epidural, intracerebral, intracerebroventricular, epicutaneous, sublingual, buccal extra-amniotic, nasal, intraarterial, intracardiac, intracavernous, intradermal, intralesional, intramuscular, intraocular, intraosseous, intraperitoneal, intrathecal, intrauterine, intravaginal, intravenous, intravesical, intravitreal, subcutaneous, transdermal, perivascular, transmucosal, and a combination thereof.

The present disclosure encompasses the use of fibroblast cells possessing regenerative properties, tissues associated with the cells possessing regenerative properties, and products derived from cells possessing regenerative properties for the treatment of cachexia or inflammation related thereto. In one embodiment, cancer-associated cachexia or HIV-associated cachexia (for example) are treated by administration of cells or tissues possessing regenerative cells that have been modified to allow for administration, while retaining properties associated with cellular regeneration. The administration may be in a manner that in some cases is intravenous, subcutaneous or intramuscular.

In embodiments of the disclosure, there are methods for ameliorating cachexia, or inflammation related thereto, in an individual, comprising the steps of: a. culturing a population of fibroblasts under suitable conditions to induce production of immune-modulating activity, thereby producing immune-modulating fibroblasts; and b. administering an effective amount of said immune-modulating fibroblasts to the individual. In specific embodiments, the cachexia, or inflammation related thereto, is initiated by a chronic inflammatory condition, such as is selected from the group consisting of chronic infection, tuberculosis, HIV/AIDS, cancer, coeliac disease, chronic obstructive pulmonary disease, multiple sclerosis, rheumatoid arthritis, congestive heart failure, familial amyloid polyneuropathy, mercury poisoning (acrodynia), Crohn's disease, untreated/severe type 1 diabetes mellitus, anorexia nervosa, hormonal deficiency, and a combination thereof. In specific embodiments, the chronic inflammatory condition is associated with the elevation of one or more detectable markers, such as detectable markers selected from the group consisting of C-reactive protein, Interleukin-1, Interleukin-6, TNF-alpha, and a combination thereof. In specific embodiments, fibroblasts are derived from amniotic fluid, such as amniotic fluid selected from the species from the group consisting of humans, primates, dogs, cats, goats, elephants, cattle, horses, pigs, mice, rabbits, and a combination thereof. The fibroblasts may express detectable levels of one or more markers selected from the group consisting of SSEA3, SSEA4, Tra-1-60, Tra-1-81, Tra-2-54, Oct-4, CD13, CD44, CD49b, CD105, aminopeptidase N, and a combination thereof. In specific embodiments, the ibroblasts do not express detectable levels of SSEA1.
10. The method of any one of claims 1-10, wherein said fibroblasts are administered to the individual affected by cachexia, or inflammation related thereto, by one or more routes selected from the group consisting of epidural, intracerebral, intracerebroventricular, epicutaneous, sublingual, buccal extra-amniotic, nasal, intraarterial, intracardiac, intracavernous, intradermal, intralesional, intramuscular, intraocular, intraosseous, intraperitoneal, intrathecal, intrauterine, intravaginal, intravenous, intravesical, intravitreal, subcutaneous, transdermal, perivascular, transmucosal, and a combination thereof.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWING

For a more complete understanding of the present disclosure, reference is now made to the following descriptions taken in conjunction with the accompanying drawing.

FIG. 1. Treatment of cachexia using skin foreskin fibroblasts. The weight of particular mice exposed to different conditions was measured. From left to right for each of the groupings of four bars: 1) control mice (Ctrl group) injected subcutaneously with PBS in the right flank of axilla; 2) cachectic mice (CA group) injected subcutaneously with 1×10⁶ LLC cells in the right flank of axilla; 3) cachectic mice treated with 100,000 foreskin fibroblasts (Cachexia+Fibroblasts (1)); and 4) 200,000 foreskin fibroblasts (Cachexia+Fibroblasts (2)).

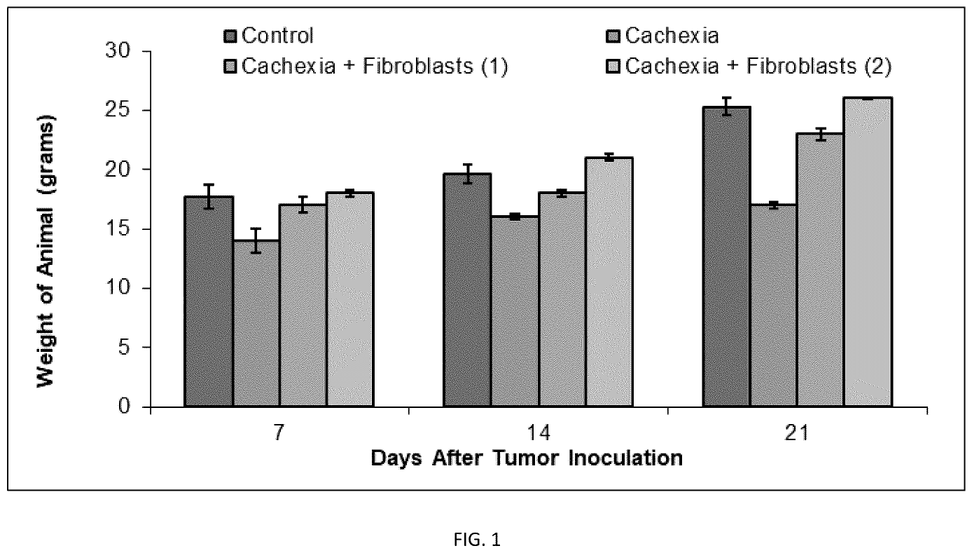

### DETAILED DESCRIPTION

### I. Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein embodiments which are disclosed and still obtain a like or similar result without departing from the spirit and scope of the disclosure. The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

“Subject” and “patient” and “individual” refer to either a human or non-human, such as primates, mammals, and vertebrates. In particular embodiments, the subject is a human, dog, cat, horse, cow, and so forth.

“Treating” or treatment of a disease or condition refers to executing a protocol, which may include administering one or more drugs or therapies (including cells) to a patient, in an effort to alleviate at least one sign or symptom of the disease. Desirable effects of treatment include decreasing the rate of disease progression, ameliorating or palliating the disease state, delaying the onset of at least one symptom, and remission or improved prognosis. Alleviation can occur prior to signs or symptoms of the disease or condition appearing, as well as after their appearance, or both. Thus, “treating” or “treatment” may include “preventing” or “prevention” of disease or undesirable condition. In addition, “treating” or “treatment” does not require complete alleviation of one or more signs or symptoms, does not require a cure, and specifically includes protocols that have only a marginal effect on the patient.

As used herein, “prevent,” and similar words such as “prevented,” “preventing” etc., indicate an approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

Cachexia refers to the unintended loss of weight associated with cancer, infectious disease and other catabolic states that cannot be reversed with nutritional support. Cachexia frequently accompanies the terminal course in patients suffering from various chronic diseases such as end-stage chronic heart failure, advanced cancer, chronic kidney disease, chronic obstructive pulmonary disease (COPD), HIV, and rheumatoid arthritis, for example. Aside from excessive weight loss, additional symptoms include anorexia, malabsorption, nausea, asthenia, neuroendocrine changes, immune system function impairment, and disruption of energy metabolism. Several methods for treating cachexia have been suggested, however, the focus has been on developing therapies directed at the underlying illness. To date, no direct treatment for cachexia is available.

Recently, there has been some level of success in better understanding the multifactorial nature of cachexia, particularly with the role of inflammatory mediators in the initiation and perpetuation of this condition. Much of this knowledge stems from earlier studies implicating bacterial lipopolysaccharides (LPS), or endotoxins, in the pathophysiological manifestations of bacterial infection, sepsis and critical illness. Recently, it was observed that cytokines derived from the macrophage/monocyte lineage can mimic the effects of many endotoxins and are central mediators responsible for the clinical phenotype of cachexia.

The present disclosure pertains to the use of fibroblasts as a means of reducing cachexia in an individual in need of therapy. More particularly, the disclosure pertains to the use of fibroblasts in reducing cachexia or inflammation-associated cachexia. More particularly, the disclosure provides means of augmenting activity of certain cells to prevent or treat cachexia in a patient in need of therapy. The treated individual may be an aging patient, the individual may be a cancer patient, the individual may be a patient infected with viral or other infections and/or the individual may be a patient with a medical disorder or disease having cachexia as a symptom. The individual may be of any age, gender, or race.

In particular embodiments, the disclosure provides for administration of regenerative cells, in one embodiment, fibroblasts, for the inhibition of cachexia or reduction in severity of at least one symptom of cachexia and/or delay in onset of cachexia. In specific embodiments, upon administration of an effective amount of fibroblasts to an individual, the fibroblasts are capable of suppressing tumor-derived molecules capable of inducing muscle atrophy and immune inhibition.

Embodiments of the disclosure include methods for ameliorating cachexia, or inflammation related thereto, in an individual, comprising the steps of optionally culturing a population of fibroblasts under suitable conditions to induce production of immune-modulating activity, thereby producing immune-modulating fibroblasts; and administering an effective amount of immune-modulating fibroblasts to the individual.

In particular embodiments, there are methods of increasing the weight of an individual that has cachexia, or preventing weight loss of an individual at risk for cachexia, comprising the step of providing to the individual an effective amount of fibroblasts, including immune-modulating fibroblasts. In specific embodiments, the fibroblasts express detectable levels of one or more markers selected from the group consisting of SSEA3, SSEA4, Tra-1-60, Tra-1-81, Tra-2-54, Oct-4, CD13, CD44, CD49b, CD105, aminopeptidase N, and a combination thereof.

The disclosure encompasses fibroblasts of any kind, for example, foreskin-derived fibroblasts, that are capable of secreting factors that inhibit inflammation, and thereby suppress the ability of the host body to induce muscle wasting. The cells may be immortal in culture, maintain euploidy for >1 year in culture, share one or more markers with human embryonic stem cells, and/or may be capable of differentiating into all three germ layers of the developing embryo: Endoderm, Mesoderm and Ectoderm.

In one embodiment, fibroblasts are derived from amniotic fluid, wherein the cells are extracted from the amnion harvested during the second trimester of human pregnancies. It is known that amniotic fluid comprises multiple morphologically-distinguishable cell types, and the majority of the cells are prone to senescence and are lost from cultures. In one embodiment, fibronectin-coated plates and culture conditions described in U.S. Pat. No. 7,569,385 (incorporated by reference herein in its entirety) are used to grow cells from amniotic fluid harvests from normal 16-18 week pregnancies. The cells of the disclosure may be of fetal origin, and have a normal diploid karyotype. Growth of the amniotic fluid-derived fibroblasts as described in the disclosure for use in neurological ischemic conditions results in cells that are multipotent, as several main cell types have been derived from them. As used herein, the term “multipotent” refers to the ability of amniotic fluid regenerative cells to differentiate into several main cell types. The amniotic fluid derived fibroblasts may also be propagated under specific conditions to become “pluripotent.” The term “pluripotent stem cells” describes stem cells that are capable of differentiating into any type of body cell, when cultured under conditions that give rise to the particular cell type. The amniotic fluid regenerative cells may be isolated from humans. However, the amniotic fluid regenerative cells may be isolated in a similar manner from other species. Examples of species that may be used to derive the amniotic fluid regenerative cells include but are not limited to mammals, humans, primates, dogs, cats, goats, elephants, endangered species, cattle, horses, pigs, mice, rabbits, and the like.

An amniotic fluid-derived fibroblast cell can be recognized by their specific cell surface proteins or by the presence of specific cellular proteins, in at least specific cases. Typically, specific cell types have specific cell surface proteins. These surface proteins can be used as “markers” to determine or confirm specific cell types. Typically, these surface markers can be visualized using antibody-based technology or other detection methods. Surface markers of the isolated amniotic fluid-derived fibroblasts cells extracted from independently-harvested amniotic fluid samples were tested for a range of cell surface and other markers, using monoclonal antibodies and FACS analysis. These cells can be characterized by the following cell surface markers: SSEA3, SSEA4, Tra-1-60, Tra-1-81, Tra-2-54, or a combination thereof. The fibroblast cells can be distinguished from mesenchymal cells in that the mesenchymal cells do not express the cell surface marker SSEA1. Additionally, fibroblasts may express the stem cell transcription factor, Oct-4. The fibroblast cells can be recognized by the presence of at least one, or at least two, or at least three, or at least four, or at least five, or at least six, or all of the following cellular markers, in some cases: SSEA3, SSEA4, Tra-1-60, Tra-1-81, Tra-2-54 and/or Oct-4, in specific embodiments.

In certain embodiments, fibroblasts of the disclosure in culture express very little or no SSEA-1 marker. In addition to the embryo stem cell markers SSEA3, SSEA4, Tra1-60, Tral-81, Tra2-54, and Oct-4, the amniotic fluid-derived fibroblasts also expressed high levels of the cell surface antigens that are normally found on human mesenchymal stem cells, but not normally on human embryo stem cells (M F Pittinger et al., Science 284:143-147, 1999; S Gronthos et al., J. Cell Physiol. 189:54-63, 2001). This set of markers includes CD13(99.6%) aminopeptidase N, CD44 (99.7%) hyaluronic acid-binding receptor, CD49b (99.8%) collagen/laminin-binding integrin alpha2, and CD105 (97%) endoglin. Presence of both the embryonic stem cell markers and the hMSC markers on the fibroblast cell cultures indicates that amniotic fluid-derived fibroblast cells, grown and propagated as described here, represent a novel class of human stem cells.

In some embodiments of the disclosure, at least about 90%, 91%, 92%, 93%, 94%, 95%, 96%, 97%, 98%, 99%, or 100% of fibroblast cells in a culture express CD13. In some embodiments, at least about 90%, 91%, 92%, 93%, 94%, 95%, 96%, 97%, 98%, 99%, or 100% of the cells in the culture express CD44. In some embodiments of the disclosure, a range from at least about 90%, 91%, 92%, 93%, 94%, 95%, 96%, 97%, 98%, 99%, or 100% of the cells in the culture express CD49b. In certain embodiments of the disclosure, at least about 90%, 91%, 92%, 93%, 94%, 95%, 96%, 97%, 98%, 99%, or 100% of the cells in the culture express CD105. In certain embodiments of the disclosure, at least about 90%, 91%, 92%, 93%, 94%, 95%, 96%, 97%, 98%, 99%, or 100% of the cells in the culture express SSEA3. In certain embodiments of the disclosure, at least about 90%, 91%, 92%, 93%, 94%, 95%, 96%, 97%, 98%, 99%, or 100% of the cells in the culture express SSEA4. In some embodiments of the disclosure, at least about 90%, 91%, 92%, 93%, 94%, 95%, 96%, 97%, 98%, 99%, or 100% of the cells in the culture express Tra-1-60. In particular embodiments of the disclosure, at least about 90%, 91%, 92%, 93%, 94%, 95%, 96%, 97%, 98%, 99%, or 100% of the cells in the culture express Tra-1-81. In certain embodiments of the disclosure, at least about 90%, 91%, 92%, 93%, 94%, 95%, 96%, 97%, 98%, 99%, or 100% of the cells in the culture express Tra-2-54. In particular embodiments of the disclosure, at least about 90%, 91%, 92%, 93%, 94%, 95%, 96%, 97%, 98%, 99%, or 100% of the cells in the culture express Oct-4. In certain embodiments of the disclosure, at least about 90%, 91%, 92%, 93%, 94%, 95%, 96%, 97%, 98%, 99%, or 100% of the cells in the culture express aminopeptidase N.

A “nutrient medium” is a medium for culturing cells containing nutrients that promote proliferation and may be utilized in embodiments of the disclosure. The nutrient medium may comprise any of the following in an appropriate combination: isotonic saline, buffer, amino acids, antibiotics, serum or serum replacement, and exogenously added factors.

General methods relating to stem cell differentiation techniques that may be useful for differentiating the amniotic fluid regenerative cells into fibroblasts of this disclosure can be found in general texts such as: Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998); and in Stem cell biology (L. M. Reid, Curr. Opinion Cell Biol. 2:121, 1990), each of which is incorporated by reference herein in its entirety.

In one embodiment of the disclosure, fibroblast cells are used to treat cachexia associated with aging. In another embodiment, cachexia-associated factors are used to guide the dose of fibroblasts provided to a patient in which cachexia is aimed to be treated. In another embodiment, fibroblasts used comprise amniotic fluid-derived fibroblast cells, and in some cases the fibroblast cells are administered intravenously. In another embodiment, amniotic membrane-derived fibroblast cells are administered. Amniotic membranes may be used as a source of fibroblast cells by direct administration. In one embodiment fibroblasts are administered a concentration of 10-200 million cells intravenously.

Administration of cachexia-inhibiting cells to a mammalian patient can be by any route, including but not limited to intravenous, intradermal, transdermal, subcutaneous, intramuscular, inhalation (e.g., via an aerosol), buccal (e.g., sub-lingual), topical (i.e., both skin and mucosal surfaces, including airway surfaces), intrathecal, intraarticular, intraplural, intracerebral, intra-arterial, intraperitoneal, oral, intralymphatic, intranasal, rectal or vaginal administration, by perfusion through a regional catheter, or by direct intralesional injection.

In a particular embodiment, the compositions of the disclosure are administered by intravenous push or intravenous infusion given over defined period (e.g., 0.5 to 2 hours). The compositions of the disclosure can be delivered by peristaltic means or in the form of a depot, although a suitable route in any given case will depend, as is well known in the art, on such factors as the species, age, gender and overall condition of the subject, the nature and severity of the condition being treated and/or on the nature of the particular composition (i.e., dosage, formulation) that is being administered. In particular embodiments, the route of administration is via bolus or continuous infusion over a period of time, once or twice a week.

In particular embodiments, the route of administration is by subcutaneous injection given in one or more sites (e.g., thigh, waist, buttocks, arm), optionally once or twice weekly. In one embodiment, the compositions, and/or methods of the disclosure are administered on an outpatient basis. Those skilled in the art will appreciate that dosages can be selected based on a number of factors including the age, sex, species and condition of the subject (e.g., activity of cachexia or inflammation related thereto), the desired degree of cachexia or inflammation related thereto to be treated can be determined by one of skill in the art. For example, effective amounts of the compositions of the disclosure may be extrapolated from dose-response curves derived from in vitro test systems or from animal model (e.g. the cotton rat or monkey) test systems.

Examples of dosing regimens that can be used in the methods of the disclosure include, but are not limited to, daily, three times weekly (intermittent), weekly, or every 14 days. In certain embodiments, dosing regimens include, but are not limited to, monthly dosing or dosing every 6-8 weeks. Those skilled in the art will appreciate that dosages are generally higher and/or frequency of administration greater for initial treatment as compared with maintenance regimens.

In some embodiments, an individual at the onset of a medical condition that is associated with cachexia begins being subjected to methods of the disclosure. The methods of the disclosure may or may not be provided to the individual at substantially the same time as one or more treatments for the medical condition. In some cases, the individual is subjected to methods of the disclosure when a symptom of cachexia becomes detectable. The fibroblasts of the disclosure may be provided to the individual before, during, and/or after any treatment for the medical condition.

Embodiments of the disclosure include methods of treating cachexia or inflammation related thereto, comprising the steps of optionally identifying an individual suffering from weight loss or cachexia; and administering to the individual an effective amount of (1) a fibroblast cell population; (2) a tissue comprising a fibroblast cell population, and/or (3) products produced by a fibroblast cell population and/or a tissue comprising a fibroblast cell population. The cachexia may or may not be associated with muscle wasting. The cachexia may or may not be associated with a neoplasia; an inflammatory condition; a chronic infection; tuberculosis; or HIV, for example. In at least some cases, an individual exhibits an elevation of one or more markers associated with chronic inflammation. An example of a marker associated with chronic inflammation includes an elevation of C-reactive Protein, interleukin-1, interleukin-6, and/or TNF-alpha, for example in peripheral blood as compared to age-matched controls. The fibroblasts may be derived from any source, but in specific embodiments they are derived from foreskin. In at least some cases, the fibroblasts are capable of adhering to plastic. The fibroblasts may be administered to the individual in the form of a morselized amniotic membrane tissue that may or may not be treated in a manner to stimulate upregulation of growth factor production, said growth factors stimulatory of anti-cachectic activities. In specific cases, such treatment comprises exposure to hypoxic conditions, acidic conditions, hypotonic conditions, or a combination thereof). The fibroblasts may or may not be administered in the form of a single cell suspension.

In particular methods of the disclosure, fibroblasts are utilized in an individual in need thereof to prevent, delay the onset of, or reduce the severity of muscle wasting (with or without accompanying cachexia), metabolic abnormalities, and multiple organ failure. When there is a delay in the onset of muscle wasting, cachexia, metabolic abnormalities, and/or multiple organ failure, the delay may be in the order of weeks, years, or months, for example.

The amount of any types of cells for administration to an individual may depend on the severity of the cachexia to be treated and/or of the type of cells to be injected for the treatment. The cells may be prepared for administration in a pharmaceutically acceptable carrier, for example a sterile saline isotonic solution. In some embodiments, the pharmaceutically acceptable carrier may comprise one or more additional agents, such as FAS ligand, IL-2R, IL-1 Ra, IL-2, IL-4, IL-8, IL-10, IL-20, IL-35, HLA-G, PD-L1, I-309, IDO, iNOS, CD200, Galectin 3, sCR1, arginase, PGE-2, aspirin, atorvastatin, fluvastatin, lovastatin, pravastatin, rosuvastatin, simvastatin, pitavastatin, n-acetylcysteine, rapamycin, IVIG, naltrexone, TGF-beta, VEGF, PDGF, CTLA-4, anti-CD45RB antibody, hydroxychloroquine, leflunomide, auranofin, dicyanogold, sulfasalazine, methotrexate, glucocorticoids, etanercept, adalimumab, abatacept, anakinra, certolizumab, Etanercept-szzs, golimumab, infliximab, rituximab, tocilizumab, cyclosporine, IFN-gamma, everolimus, rapamycin, VEGF, FGF-1, FGF-2, angiopoietin, HIF-1-alpha, or a combination thereof.

In one embodiment of the disclosure, fibroblasts are administered to a subject by any suitable route, including by injection (such as intramuscular injection), including in hypoxic areas. Suitable routes include intravenous, subcutaneous, intrathecal, oral, intrarectal, intrathecal, intra-omentral, intraventricular, intrahepatic, and intrarenal.

In certain embodiments, fibroblasts may be derived from tissues comprising skin, heart, blood vessels, bone marrow, skeletal muscle, liver, pancreas, brain, adipose tissue, foreskin, placental, and/or umbilical cord. In specific embodiments, the fibroblasts are placental, fetal, neonatal or adult or mixtures thereof.

The number of administrations of cells to an individual will depend upon the factors described herein at least in part and may be optimized using routine methods in the art. In specific embodiments, a single administration is required. In other embodiments, a plurality of administration of cells is required. It should be appreciated that the system is subject to variables, such as the particular need of the individual, which may vary with time and circumstances, the rate of loss of the cellular activity as a result of loss of cells or activity of individual cells, and the like. Therefore, it is expected that each individual could be monitored for the proper dosage, and such practices of monitoring an individual are routine in the art.

In one embodiment of the disclosure, cells (such as fibroblasts) are cultured ex vivo using means known in the art for preserving viability and proliferative ability of the cells. In specific embodiments for fibroblasts, there may be modification of known culture techniques to achieve one or more desired effects for the cells, such as to decrease visibility of fibroblasts to a recipient immune system. In one embodiment, cells (for example, fibroblasts) are cultured in conditions that lack one or more xenogeneic components, such as fetal calf serum. In specific embodiments, the disclosure encompasses the substitution of fetal calf serum with human platelet rich plasma, platelet lysate, umbilical cord blood serum, autologous serum, and/or defined cytokine mixes as an additional feature, for example to reduce the immunogenicity of the cells (such as fibroblasts).

### EXAMPLE

The following example is included to demonstrate certain non-limiting aspects of the disclosure. It should be appreciated by those of skill in the art that the techniques disclosed in the examples that follow represent techniques discovered by the inventors to function well in the practice of the disclosed subject matter. However, those of skill in the art should, in light of the present disclosure, appreciate that many changes can be made in the specific embodiments that are disclosed and still obtain a like or similar result without departing from the spirit and scope of the disclosed subject matter.

### Example 1

### Treatment of Cachexia Using Skin Foreskin Fibroblasts

Lewis lung carcinoma (LLC) cells were obtained from American Type Culture Collection (ATCC) and were cultured at 37 °C with 5% CO₂ using Dulbecco's modified Eagle's medium containing 10% fetal bovine serum (Gibco; Thermo Fisher Scientific, Waltham, MA, USA) and 1% penicillin/streptomycin (Boster Biological Technology, Wuhan, China). Before tumor inoculation, LLC cells were centrifuged at 1000 rpm at 4 °C for 5 min and resuspended in phosphate buffer saline (PBS).

Specific pathogen-free C57BL/6 mice aged 5-6 weeks were housed free for chow and water in the condition of stable temperature and humidity with a regular 12-h light-dark cycle under the specific pathogen-free environment. C57BL/6 mice were randomly assigned into three groups (7 mice per group): 1) control mice (Ctrl group) injected subcutaneously with PBS in the right flank of axilla; 2) cachectic mice (CA group) injected subcutaneously with 1×10⁶ LLC cells in the right flank of axilla; 3) cachectic mice treated with 100,000 foreskin fibroblasts (ATCC) (Cachexia+Fibroblasts (1)); and 4) 200,000 foreskin fibroblasts (Cachexia+Fibroblasts (2)). The body weight of each mouse and cumulative food intake (combined of all seven mice in each group) were monitored every 3 days following tumor implantation.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method for ameliorating cachexia, or inflammation related thereto, in an individual, comprising:
administering an effective amount of fibroblasts to the individual.

2. The method of claim 1, wherein said cachexia, or inflammation related thereto, is initiated by a chronic inflammatory condition.

3. The method of claim 2, wherein said chronic inflammatory condition is selected from the group consisting of chronic infection, tuberculosis, HIV/AIDS, cancer, coeliac disease, chronic obstructive pulmonary disease, multiple sclerosis, rheumatoid arthritis, congestive heart failure, familial amyloid polyneuropathy, mercury poisoning (acrodynia), Crohn's disease, untreated/severe type 1 diabetes mellitus, anorexia nervosa, hormonal deficiency, and a combination thereof.

4. The method of claim 3, wherein said chronic inflammatory condition is associated with the elevation of one or more detectable markers.

5. The method of claim 4, wherein said detectable markers are selected from the group consisting of C-reactive protein, Interleukin-1, Interleukin-6, TNF-alpha, and a combination thereof.

6. The method of claim 1, wherein said fibroblasts are derived from amniotic fluid.

7. The method of claim 6, wherein said fibroblasts derived from amniotic fluid are selected from the species from the group consisting of humans, primates, dogs, cats, goats, elephants, cattle, horses, pigs, mice, rabbits, and a combination thereof.

8. The method of claim 1, wherein said fibroblasts express detectable levels of one or more markers selected from the group consisting of SSEA3, SSEA4, Tra-1-60, Tra-1-81, Tra-2-54, Oct-4, CD13, CD44, CD49b, CD105, aminopeptidase N, and a combination thereof.

9. The method of claim 1, wherein said fibroblasts do not express detectable levels of SSEA1.

10. The method of claim 1, wherein said fibroblasts are administered to the individual affected by cachexia, or inflammation related thereto, by one or more routes selected from the group consisting of epidural, intracerebral, intracerebroventricular, epicutaneous, sublingual, buccal extra-amniotic, nasal, intraarterial, intracardiac, intracavernous, intradermal, intralesional, intramuscular, intraocular, intraosseous, intraperitoneal, intrathecal, intrauterine, intravaginal, intravenous, intravesical, intravitreal, subcutaneous, transdermal, perivascular, transmucosal, and a combination thereof.

11. A method of increasing the weight of an individual with cachexia or at risk of having cachexia, comprising the step of administering an effective amount of fibroblasts to the individual.

12. The method of claim 11, wherein the individual is at risk for having cachexia has chronic infection, tuberculosis, HIV/AIDS, cancer, coeliac disease, chronic obstructive pulmonary disease, multiple sclerosis, rheumatoid arthritis, congestive heart failure, familial amyloid polyneuropathy, mercury poisoning (acrodynia), Crohn's disease, untreated/severe type 1 diabetes mellitus, anorexia nervosa, hormonal deficiency, or a combination thereof.

13. The method of claim 11, wherein the fibroblasts express one or more markers selected from the group consisting of SSEA3, SSEA4, Tra-1-60, Tra-1-81, Tra-2-54, Oct-4, CD13, CD44, CD49b, CD105, aminopeptidase N, and a combination thereof.

14. The method of claim 11, further comprising the step of obtaining fibroblasts that express one or more markers selected from the group consisting of SSEA3, SSEA4, Tra-1-60, Tra-1-81, Tra-2-54, Oct-4, CD13, CD44, CD49b, CD105, aminopeptidase N, and a combination thereof.

15. The method of claim 11, wherein said fibroblasts do not express detectable levels of SSEA1.

16. The method of claim 11, wherein said fibroblasts are administered to the individual affected by cachexia, or inflammation related thereto, by one or more routes selected from the group consisting of epidural, intracerebral, intracerebroventricular, epicutaneous, sublingual, buccal extra-amniotic, nasal, intraarterial, intracardiac, intracavernous, intradermal, intralesional, intramuscular, intraocular, intraosseous, intraperitoneal, intrathecal, intrauterine, intravaginal, intravenous, intravesical, intravitreal, subcutaneous, transdermal, perivascular, transmucosal, and a combination thereof.
